(19) United States
(12) Patent Application Publication
(10) Pub. No.: US 2003/0185920 A1
(43) Pub. Date: Oct. 2, 2003

(54) Sebum-like preparation to be used as lipidic component in cosmetics

(76) Inventor: Siro Passi, Rome (IT)

Correspondence Address: YOUNG & THOMPSON, 745 SOUTH 23RD STREET 2ND FLOOR, ARLINGTON, VA, 22202

(21) Appl. No.: 10/343,646

(22) PCT Filed: Aug. 2, 2001
(86) PCT No.: PCT/IT01/00428

(30) Foreign Application Priority Data
Aug. 3, 2000 (IT) RM2000A000440

(51) Int. Cl. A61K 35/78; A61K 31/66; A61K 31/56

(52) U.S. Cl. 424/757; 424/769; 514/171; 514/114

(57) ABSTRACT

A preparation similar to sebum to be used as lipidic component in formulations to be administered topically is characterized in that it consits of active principale of only natural origin capable of fulfilling the requirement of meeting the average qualitative-quantitative composition of the human dermal surface lipides. In particular, it consits of substances of natural origin capable of providing triglycerides which are rich in saturated, monounsaturated and diunsaturated acids, squalene, waxes, free and esterified cholesterol in quantities which are equivalent to those present in the human sebum.

The present invention relates to formulations to be administered topically and intended to re-establish the equilibrium of lipids functional to the dermal homeostasis and altered by ageing and different dermatosis. Such formulations are characterized in that they include active principles of only natural origin capable of fulfilling the requirement of meeting the average qualitative-quantitative composition of the human dermal surface lipids.

Human dermal surface lipids have the peculiarity of being the sole lipid in the animal and vegetal kingdoms both as far as plenty of fraction and the complexity and the peculiarity of their intimate structure is concerned. The following Table 1 shows the average percentage and weight composition of dermal surface lipids (DSL) calculated on the forehead of 30 normal subjects aged between 20 and 35: the DSL are formed by 90% sebum.

TABLE 1

| Lipidic fraction | average ± SD |
|---|---|
| squalene | 12.8 ± 0.6 |
| cholesterol esters (CE) | 1.3 ± 0.2 |
| waxes (WE) | 25.6 ± 3.2 |
| triglycerides (TG) | 36.1 ± 8.4 |
| free fatty acids (FFA) | 21.6 ± 8.8 |
| free cholesterol (CH) | 1.2 ± 0.2 |
| diglycerides (DG) | 0.9 ± 0.2 |
| monoglycerides (MG) | 0.3 ± 0.2 |
| Total lipids (μg/cm²): | 195.4 ± 20.6 |

In the other dermal areas rich in oil glands (cheeks, chest, back) the percentage of the lipidic fraction is similar to that of Table 1 relating to the forehead. As it can be easily inferred, the quantity (μg/cm²) of extracted lipids varies in a proportional way as the density of the oil glands. For example, it is 122.5±21.2 μg/cm² in the chest (at the clavicula). It is known that fat represents one of the primary components of cosmetics and its function is to protect and to re-establish the equilibrium of lipids in the skin. It should be pointed out, however, that the fats used in cosmetics are almost of synthetic origin and do not fulfil at all the qualitative-quantitative composition shown in Table 1.

The present invention seeks to provide a mixture of lipids similar to sebum of clear natural origin that is quite similar to the human sebum produced by the oil glands. The application of such mixture to the skin should have the function of avoiding an excessive evaporation of water, protecting the skin, from external agents, and ensuring the softness. Sebum consists of triglycerides by about 60%, waxes (esters of fatty acids and alcohols) by 25%, squalene (an intermediate product of cholesterol synthesis) by 12%, and cholesterol and its esters by the balance. On the skin, triglycerides are hydrolyzed to diglycerides, monoglycerides and fatty acids free from lipase of the resident flora. As such fractions are actually products of the hydrolysis of triglycerides and not classes directly biosynthesized by the oil glands, they have not been used in the formulations of the invention. According to a preferred embodiment, a mixture consisting of 35-70%, preferably 50%, macadamia oil, and 5-15%, preferably 10%, avocado oil, both rich in saturated acids, monounsaturated acids and diunsaturated acids which are quite similar to dermal acids can be used as triglycerides. Still according to the present invention, 6-15%, preferably 12%, unsaponifiable product of olive oil, 18-30%, preferably 24%, jojoba wax, 0.5-3%, preferably 1.5%, free cholesterol, and 0.5-1.5%, preferably 0.5%, oleate cholesterol are able to provide squalene, waxes, free, esterified cholesterol in a quantity which is equivalent and quite similar by their structure to those present in the human sebum. A first formulation of an oil similar to sebum to be used as lipid mixture in cosmetic preparations according to the present invention is as follows:

| | |
|---|---|
| macadamia oil | 35-70% |
| jojoba oil (wax) | 18-30% |
| avocado oil | 5-15% |
| unsaponifiable product of olive oil | 6-15% |
| free cholesterol | 0.5-3% |
| oleate cholesterol | 0.5-1.5% |
| Soia phospholipids | 1-3% |
| BHT (butylhydroxytoluene) | 0.05-0.15% |

It should be noted from the experiment carried out that a preferred formulation of the described oil similar to sebum is as follows:

| Ingredients (g, ml) | % |
|---|---|
| macadamia oil | 50 |
| jojoba oil (wax) | 24.5 |
| avocado oil | 10 |
| unsaponifiable product of olive oil | |
| (squalene) | 12 |
| free cholesterol | 1.5 |
| oleate cholesterol | 0.5 |
| Soia phospholipids | 1.4 |
| BHT (butylhydroxytoluene) (preservative) | 0.1 |

1. A preparation similar to sebum to be used as lipidic component in formulations to Abe administered topically, characterized in that it consists of substances of natural origin capable of providing triglycerides which are rich in saturated, monounsaturated and diunsaturated acids, squalene, waxes, free and esterified cholesterol in quantities which are equivalent to those present in the human sebum.

2. The preparation according to claim 1, characterized in that it has the following formulation in weight percentage:

| | |
|---|---|
| macadamia oil | 35 to 70 |
| jojoba oil (wax) | 18 to 30 |
| avocado oil | 5 to 15 |
| unsaponifiable product of olive oil | 6 to 15 |
| free cholesterol | 0.5 to 3 |
| oleate cholesterol | 0.5 to 1.5 |
| Soia phospholipids | 1 to 3 |
| BHT (butylhydroxytoluene) | 0.05 to 0.15 |

3. The preparation according to claims 1 and 2, characterized in that it has the following formulation in weight percentage:

| | |
|---|---|
| macadamia oil | 50 |
| jojoba oil (wax) | 24.5 |
| avocado oil | 10 |
| unsaponifiable product of olive oil | |
| (squalene) | 12 |
| free cholesterol | 1.5 |
| oleate cholesterol | 0.5 |
| Soia phospholipids | 1.4 |
| BHT (butylhydroxyboluene) | 0.1 |
